(19) United States
(12) Patent Application Publication
(10) Pub. No.: US 2026/0062693 A1
(43) Pub. Date: Mar. 5, 2026

(54) MUTANT CREATION APPARATUS, METHOD, AND PROGRAM

(71) Applicant: NTT, Inc., Tokyo (JP)

(72) Inventors: Sosuke IMAMURA, Musashino-shi (JP); Kazuhiro TAKAYA, Musashino-shi (JP); Hidenori IWASHITA, Musashino-shi (JP); Ryu KIUCHI, Musashino-shi (JP)

(73) Assignee: NTT, Inc., Tokyo (JP)

(21) Appl. No.: 19/105,909

(22) PCT Filed: Aug. 25, 2022
(86) PCT No.: PCT/JP2022/032057

§ 371 Date: Feb. 24, 2025

(51) Int. Cl. C12N 15/01; C12M 1/42

(52) U.S. Cl. CPC C12N 15/01; C12M 35/08

(57) ABSTRACT

A mutant creation apparatus according to an embodiment includes: a mutation induction unit that irradiates an organism, or a target for creating a mutant, with a radiation or electromagnetic energy for inducing a mutation in a nucleotide sequence that causes a trait variation in the organism or introduces a chemical substance into the organism for inducing the mutation; a selection unit that selects a mutant having a desired trait from mutants of the organism obtained by the mutation induction unit; a nucleotide sequence analysis unit that analyzes a nucleotide sequence of the selected mutant; an identification unit that identifies a nucleotide sequence that provides the desired trait of the mutant in the analyzed nucleotide sequence; and a condition analysis unit that analyzes a condition under which a desired mutant of the organism is created by the mutation induction unit based on a result of identification.

### TECHNICAL FIELD

An embodiment of the present invention relates to a mutant creation apparatus, method, and program.

### BACKGROUND ART

Recombinant DNA technology or genome editing technology is employed to obtain a desired trait variation such as an increase in yield and improvement in appearance of an organism, for example, crops and other plants. An advantage of recombinant DNA technology and genome editing technology is that, as long as a target gene is determined, it is possible to obtain a desired trait variation in the target gene by CRISPR/Cas9 or the like (for example, see Non Patent Literature 1).

Irradiation of a target organism with radiations such as gamma rays, X-rays, heavy particle beams, neutron beams, and proton beams from the outside of a cell has been confirmed to induce a mutation in which one or two bases in a gene sequence are deleted or to cause an inversion or a translocation of a chromosome. In addition, there is a method of inducing a mutation by utilizing light or electromagnetic energy such as ultraviolet rays and other electromagnetic waves (see, for example, Non Patent Literature 2).

Typically, the higher the Linear Energy Transfer (LET), which represents the amount of locally applied energy, the more the radiation influences a living body to be irradiated. Neutron beams, proton beams, and heavy particle beams as high-LET radiations directly act on biomolecules and invoke ionization or excitation.

On the other hand, gamma rays and x-rays as low-LET radiations generate free radicals and reactive oxygen species through ionization or excitation of water molecules, and these reactive molecular species secondarily cause damage to biomolecules (see, for example, Non Patent Literature 3).

Comparatively, electromagnetic waves such as ultraviolet rays or light energy are referred to as non-ionizing radiations. For example, when a nucleotide sequence in a gene (deoxyribonucleic acid (DNA)) is irradiated with ultraviolet rays from the outside of a cell, adjacent thymine bases (T) in the DNA generate a thymine dimer (pyrimidine dimer), which causes damage to one strand of double stranded DNA and blocks replication or transcription of this damaged DNA, thereby inducing a mutation (see, for example, Non Patent Literature 4).

Furthermore, there is a method of mutation induction (mutagenesis) by introducing a chemical substance into a target organism from the outside of a cell. In this method, when Ethyl methanesulfonate (EMS) or the like is introduced, an ethyl group of EMS, as an alkylating agent, ethylates guanine (G). Replacing this guanine with adenine (A) causes a bond between guanine (G) and cytosine (C) to be replaced by a bond between adenine (A) and thymine (T), thereby inducing a mutation (see, for example, Non Patent Literature 5).

In this manner, the mutation induction by a radiation, electromagnetic energy, or a chemical substance is based on a phenomenon of blocking replication or transcription of a damaged gene (DNA) by an external factor.

There is another method of mutation induction which employs transposons. This method exploits the property of an enzyme referred to as transferase that allows transcription or translation with the help of various proteins in a cell (see, for example, Non Patent Literature 6).

### CITATION LIST

### Non Patent Literature

Non Patent Literature 1: Jinek M, et al: A programmable dual-RNA-guided DNA endonuclease in adaptive bacterial immunity. Science, 337:816-821, 2012

Non Patent Literature 2: Saga and Shikazono. Radiation-induced clustered DNA lesions: Repair and mutagenesis. Free radical biology and medicine. 2017; 107:125-135. doi: 10.1016/j.freeradbiomed.2016.12.008

Non Patent Literature 3: Matsumoto et al: Heavy-ion beam-induced reactive oxygen species and redox reactions. Free radical research. 2021; 55(4):450-460. doi: 10.1080/10715762.2021.1899171

Non Patent Literature 4: Ikehara and Ono, The mechanisms of UV mutagenesis. Journal of radiation research, 2011; 52(2):115-25. doi: 10.1269/jrr.10175.

Non Patent Literature 5: Sega, A review of the genetic effects of ethyl methanesulfonate. Mutation Research, 1984; 134(2-3):113-42. doi: 10.1016/0165-1110(84)90007-1

Non Patent Literature 6: Jiang SY, Ramachandran S. Natural and artificial mutants as valuable resources for functional genomics and molecular breeding. International journal of biological sciences. 2010 6(3):228-51. doi: 10.7150/ijbs.6.228

### SUMMARY OF INVENTION

### Technical Problem

In creating a mutant by recombinant DNA technology or genome editing technology, reams of genome information of a target organism is necessary, and it is required to identify a gene directly linked to a desired trait variation.

Furthermore, in order to modify or edit a gene, it is required to introduce a vector, for example, a nucleic acid and a protein, which plays a role in delivering a gene from the outside of a cell. Still further, optimal conditions for the introduction are not clarified. Under current circumstances, recombinant DNA technology and genome editing technology count on the experience or intuition of those skilled in the art and cannot be applied to all organisms.

Even though a mutation is obtained by the vector introduction, if an organism can copy a plurality of diploids or a chromosome, it is required to cultivate progeny, and then, select progeny so as to select only a desired mutant.

In addition, even though recombination of a desired gene or genome editing enables a change in gene sequence as expected, it is required to grow a target organism and check whether a desired trait variation is obtained. Therefore, even though a target gene is determined for genetic modification or editing, a desired trait is not always obtained.

In a method of irradiating a target organism with low-LET radiations such as gamma rays and X-rays, a trait variation occurs through ionization or excitation of water molecules. For this reason, the method is applicable to a limited cell and limited irradiation conditions.

When using high-LET radiations such as proton beams and heavy particle beams, although these radiations directly act on biomolecules and invoke ionization or excitation, when a target organism needs to be irradiated while being cultured in a medium such as microalgae or in water, the transmission through the medium or water is relatively low, and it is difficult to uniformly irradiate the target organism down to the inside.

With regard to neutron beams as a high-LET radiation, the transmission is relatively high, but what condition (irradiation dose, time, cycle, and the like) induces what kind of mutation is scarcely studied.

The aforementioned method for inducing a mutation in a nucleotide sequence that causes a trait variation with use of light or electromagnetic energy such as ultraviolet rays and other electromagnetic waves does not invoke ionization or excitation of biomolecules and is applicable to a limited range for mutation induction.

For example, the mutation induction by ultraviolet irradiation utilizes absorption by double bonds of a thymine base and a cytosine base in DNA. If adjacent bases are thymine bases or cytosine bases, a mutation is induced due to a covalent bond between the two bases. For this reason, this mutation induction is limited to a specific nucleotide sequence and use. In addition, it is also difficult to irradiate only a target cell or gene.

Mutation induction by introducing a chemical substance such as ethyl methanesulfonate (EMS) is a method focusing on the fact that adenine (A) and thymine (T), and guanine (G) and cytosine (C) are specifically bound (A:T pairing and G:C pairing) in base pairs of DNA. In this method, a mutation is induced by replacing these pairings. For this reason, this method depends on gene sequences, and a desired mutation is not caused in all gene sequences.

In other words, this method is employed to induce some sort of mutation in any gene sequence but is not necessarily employable if a desired trait variation is intended.

In the mutation induction by a radiation, electromagnetic energy, or a chemical substance, not only the aforementioned problems on the methods remain to be solved but also a mutant creation method after mutation induction has not been clarified.

This invention has been made in view of the aforementioned circumstances, and an object of this invention is to provide a mutant creation apparatus, method, and program for appropriately creating a mutant that causes a desired trait variation.

### Solution to Problem

A mutant creation apparatus according to an aspect of the present invention includes: a mutation induction unit that irradiates an organism, or a target for creating a mutant, with a radiation or electromagnetic energy for inducing a mutation in a nucleotide sequence that causes a trait variation in the organism or introduces a chemical substance into the organism for inducing the mutation; a selection unit that selects a mutant having a desired trait from mutants of the organism obtained by the mutation induction unit; a nucleotide sequence analysis unit that analyzes a nucleotide sequence of the mutant selected by the selection unit; an identification unit that identifies a nucleotide sequence that provides the desired trait of the mutant in the nucleotide sequence analyzed by the nucleotide sequence analysis unit; and a condition analysis unit that analyzes a condition under which a desired mutant of the organism is created by the mutation induction unit based on a result of identification by the identification unit.

A mutant creation method according to an aspect of the present invention is a method performed by a mutant creation apparatus, the method involving: irradiating an organism, or a target for creating a mutant, with a radiation or electromagnetic energy for inducing a mutation in a nucleotide sequence that causes a trait variation in the organism or introducing a chemical substance into the organism for inducing the mutation, using a mutation induction unit of the mutant creation apparatus; selecting a mutant having a desired trait from mutants of the organism obtained by the mutation induction unit, using a selection unit of the mutant creation apparatus; analyzing a nucleotide sequence of the mutant selected by the selection unit, using a nucleotide sequence analysis unit of the mutant creation apparatus; identifying a nucleotide sequence that provides the desired trait of the mutant in the nucleotide sequence analyzed by the nucleotide sequence analysis unit, using an identification unit of the mutant creation apparatus; and analyzing a condition under which a desired mutant of the organism is created by the mutation induction unit based on a result of identification by the identification unit, using a condition analysis unit of the mutant creation apparatus.

### Advantageous Effects of Invention

According to the present invention, it is possible to appropriately create a mutant that causes a desired trait variation.

### BRIEF DESCRIPTION OF DRAWINGS

FIG. 1 is a view illustrating an example of a mutant creation system according to a first embodiment of the present invention.

FIG. 2 is a flow chart illustrating procedures performed by the mutant creation system according to the first embodiment of the present invention.

FIG. 3 is a view illustrating irradiation of algae with neutrons and a distribution of neutron energy.

FIG. 4A is a view illustrating characteristics of high-energy neutrons.

FIG. 4B is a view illustrating characteristics of thermal neutrons.

FIG. 5 is a view illustrating an example of a mutant creation system according to a second embodiment of the present invention.

FIG. 6 is a flow chart illustrating procedures performed by the mutant creation system according to the second embodiment of the present invention.

FIG. 7 is a block diagram illustrating a hardware configuration of a relation analysis device according to an

### EMBODIMENT OF THE PRESENT INVENTION

### Description of Embodiments

Hereinafter, an embodiment according to the present invention will be described with reference to the drawings.

In an embodiment of the present invention, there is provided a mutant creation method after mutation induction. The method involves a step of selecting an optimal irradiation means including irradiation with neutron beams among radiations and electromagnetic energy with respect to a target genome nucleotide sequence, for example, a gene sequence or a nucleotide sequence in a region other than a gene, of a target organism to be subjected to mutation induction. The term “organism” in this embodiment includes an individual, an organ, a tissue, and a cell.

Particularly, with respect to a medium such as algae or a target organism in an aqueous solution, adding a means for irradiating neutron beams makes it possible to exert an effect uniformly on the irradiation target from the outside of a cell and also provides optimal conditions for neutron beam irradiation which have not been clarified in the related art.

An embodiment of the present invention also provides a method for selecting an appropriate medium and conditions for irradiating a target organism with a radiation or electromagnetic energy in order to obtain a desired trait variation. Accordingly, it is possible to cause various levels of random damage, that is, from a level that gives damage to one base to a level that gives damage to a plurality of genes, thereby causing various mutations simultaneously. In addition, like the creation of a mutant by recombinant DNA technology or genome editing technology, selecting a mutant that exhibits a desired trait variation from various mutants eliminates the need to identify a genome nucleotide sequence directly linked to the desired trait variation and the need to evaluate the trait variation after a mutation. Accordingly, it is possible to significantly shorten the total time needed to create a mutant that causes the desired trait variation.

### (1) First Embodiment

Hereinafter described in a first embodiment is the method for inducing a mutation by irradiating algae with neutrons.

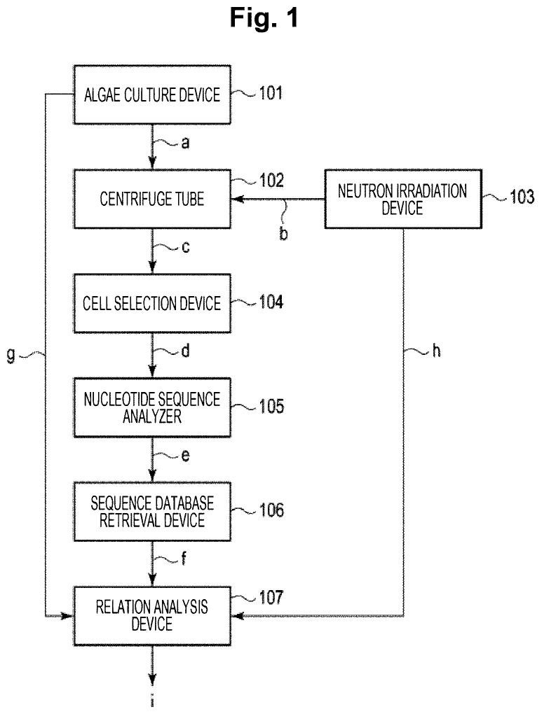

FIG. 1 is a view illustrating an example of a mutant creation system according to the first embodiment of the present invention.

As illustrated in FIG. 1, the mutant creation system according to the first embodiment includes an algae culture device 101, a centrifuge tube 102, a neutron irradiation device 103, a cell selection device 104, a nucleotide sequence analyzer 105, a sequence database (DB) retrieval device 106, and a relation analysis device 107. The sequence database retrieval device 106 includes, for example, a sequence database of a non-volatile memory. All or part of the elements of this system may be assembled to constitute a mutant creation apparatus. Among the elements of the mutant creation system, for example, the neutron irradiation device 103, the cell selection device 104, the nucleotide sequence analyzer 105, the sequence database retrieval device 106, and the relation analysis device 107 may be assembled to constitute the mutant creation apparatus.

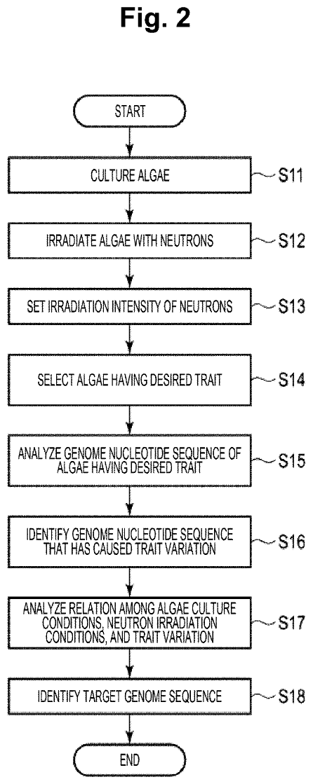

FIG. 2 is a flow chart illustrating procedures performed by the mutant creation system according to the first embodiment of the present invention.

In step S11, the algae culture device 101 is used to culture algae. Herein, the algae culture device 101 prepares algae in various states.

The various states include the number of cells contained per unit volume of a culture solution, the nutritional status of a medium, the wavelength and intensity of light, growth phases such as log phase and stationary phase, and phases of the cell cycle such as Gap 1 (G1) phase, Gap 2 (G2) phase, synthesis (S) phase, and mitotic (M) phase.

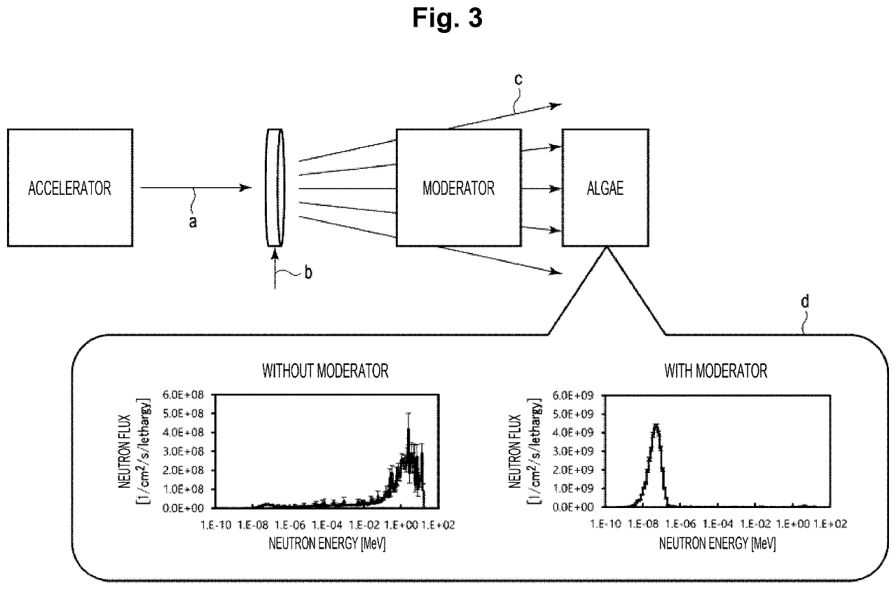

FIG. 3 is a view illustrating irradiation of algae with neutrons and a distribution of neutron energy.

In step S12, a target for neutron production (symbol b in FIG. 3), for example, beryllium (Be), tungsten (W), lithium (Li), lead (Pb), and mercury (Hg) is irradiated with charged particles (symbol a in FIG. 3) accelerated by an accelerator. The aforementioned cultured algae is irradiated with neutrons (symbol c in FIG. 3) from this target for neutron production.

For example, an algae culture solution (symbol a in FIG. 1) is transferred to the centrifuge tube 102 having a Capacity of, for example, 50 ml, and the neutron irradiation device 103 is set to irradiate the algae with neutrons (symbol b in FIG. 1).

Neutrons during the production are mainly composed of high-energy neutrons, but using a hydrogen-containing substance such as water, heavy water, polyethylene, and methane as a moderator slows the high-energy neutrons to thermal neutrons, or low-energy neutrons.

Accordingly, adjusting the position and thickness of the moderator enables adjustment of a ratio between the high-energy neutrons and the thermal neutrons, thereby enabling an energy distribution of various neutrons (symbol d in FIG. 3).

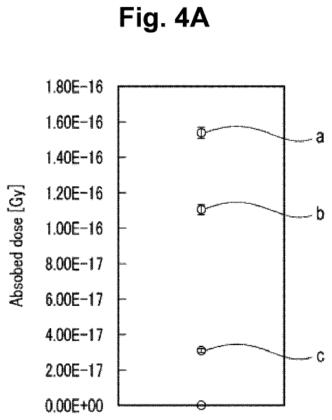

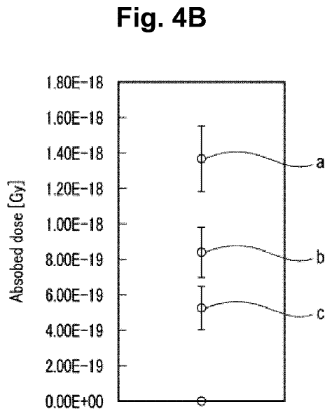

FIG. 4A is a view illustrating characteristics of high-energy neutrons. FIG. 4B is a view illustrating characteristics of thermal neutrons.

Changing the neutron energy makes it possible to change the type of particles that cause a mutation, that is, particles that contribute to the total absorbed dose (symbol a in FIGS. 4A and 4B). If the particles are high-energy neutrons, protons (symbol b in FIG. 4A) assume a key role but not electrons (symbol c in FIG. 4A). Comparatively, if the particles are thermal neutrons, both electrons (symbol b in FIG. 4B) and neutrons (symbol c in FIG. 4B) assume a key role.

In step S13, the neutron irradiation device 103 sets an irradiation intensity of neutrons with respect to the algae culture solution. Since the irradiation intensity is proportional to the current value of accelerated particle beams and the irradiation time and inversely proportional to the square of the distance, changing these conditions enables adjustment of the absorbed dose [Gy] with respect to the algae.

In step S14, from the algae culture solution (symbol c in FIG. 1) containing mutant cells obtained by the neutron irradiation, an algal cell having a desired trait is isolated (symbol d in FIG. 1).

With regard to this isolation, for example, the cell selection device 104, or a flow cytometer, is used to sort (select) each cell using the following indices: (1) the size of a cell, (2) chlorophyll fluorescence, and (3) the fluorescence intensity emitted by a fluorescent dye (boron-dipyrromethene (BODIPY) or Nile Red is used for fat and oil) that specifically stains a target substance.

In step S15, the algal cell isolated in step S14 is subjected to analysis (mapping) of its genome nucleotide sequence (which may be simply referred to as nucleotide sequence). With regard to this analysis, the nucleotide sequence analyzer 105 such as a next-generation sequencer is used to analyze the genome nucleotide sequence of the algae and to output results of the analysis (symbol e in FIG. 1).

In step S16, the analyzed genome nucleotide sequence is supplied to the sequence database retrieval device 106, and the sequence database retrieval device 106 compares the analyzed genome nucleotide sequence with a genome nucleotide sequence of the algal cell before the neutron irradiation, that is, before a mutation, which is stored in the internal sequence database. Accordingly, a mutated sequence of the genome nucleotide sequence caused by the neutron irradiation is identified, thereby identifying a mutation pattern, that is, a genome nucleotide sequence that has caused a trait variation in the isolated algal cell.

In step S17, the relation analysis device 107 analyzes the relation among information on the identified mutated sequence, information on the identified genome nucleotide sequence that has caused a trait variation in the isolated cell (symbol f in FIG. 1), and conditions including conditions for mutation induction, that is, conditions for irradiating neutrons by the neutron irradiation device 103 (symbol h in FIG. 1) and conditions for culturing algae by the algae culture device 101 (symbol g in FIG. 1).

The relation analysis device 107 performs analysis of principal components and statistic correlation analysis, thereby extracting a neutron irradiation condition and an algae culture condition for obtaining a desired trait variation in the algal cell.

Furthermore, in step S18, the relation analysis device 107 identifies a target genome sequence for obtaining a similar mutation by conventional genome editing based on the mutation in the genome nucleotide sequence for obtaining the desired trait variation and outputs results of the extraction and identification (symbol i in FIG. 1).

Conventional genome editing takes an immense amount of time to identify a target genome sequence that causes a desired trait variation and to determine the trait variation after genome editing. In contrast, isolating algae that exhibits a desired trait variation makes it possible to easily clarify a target genome sequence and its trait variation. Accordingly, it is possible to greatly reduce the time required for creating a valuable mutant.

In particular, appropriately adjusting the irradiation intensity of neutrons in steps S12 and S13 makes it possible to attempt mutation induction that is expected to cause various levels of trait variations, that is, from a level that causes a mutation in one or two nucleotide sequences to a level that causes a mutation in several genes. Accordingly, it is possible to achieve many different trait variations, which greatly increases the probability of discovering a target genome sequence optimal for a desired trait variation.

### (2) Second Embodiment

Hereinafter described in a second embodiment is a method for inducing a mutation in a target organism. In the method, a mutation is induced by selecting an optimal method of mutation induction from various types of methods including neutron irradiation, ultraviolet irradiation, heavy particle beam irradiation, and mutagenic treatment.

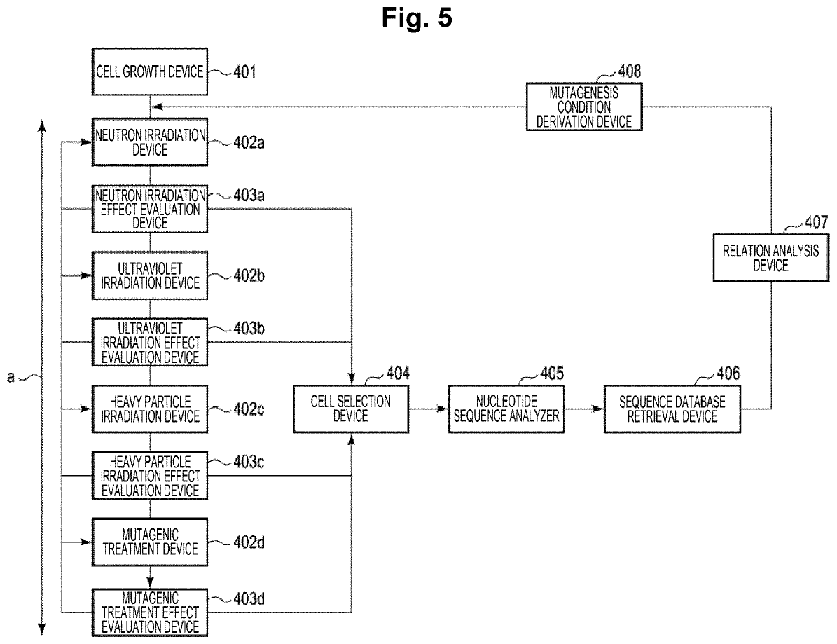

FIG. 5 is a view illustrating an example of a mutant creation system according to the second embodiment of the present invention.

As illustrated in FIG. 5, the mutant creation system according to the second embodiment includes a cell growth device 401, a neutron irradiation device 402a, an ultraviolet irradiation device 402b, a heavy particle irradiation device (heavy particle beam irradiation device) 402c, a mutagenic (ethyl methanesulfonate (EMS) ) treatment device 402d, a neutron irradiation effect evaluation device 403a, an ultraviolet irradiation effect evaluation device 403b, a heavy particle irradiation effect evaluation device (heavy particle beam irradiation effect evaluation device) 403c, a mutagenic treatment effect evaluation device 403d, a cell selection device 404, a nucleotide sequence analyzer 405, a sequence database retrieval device 406, a relation analysis device 407, and a mutagenesis condition derivation device 408. All or part of the elements of this system may be assembled to constitute a mutant creation apparatus.

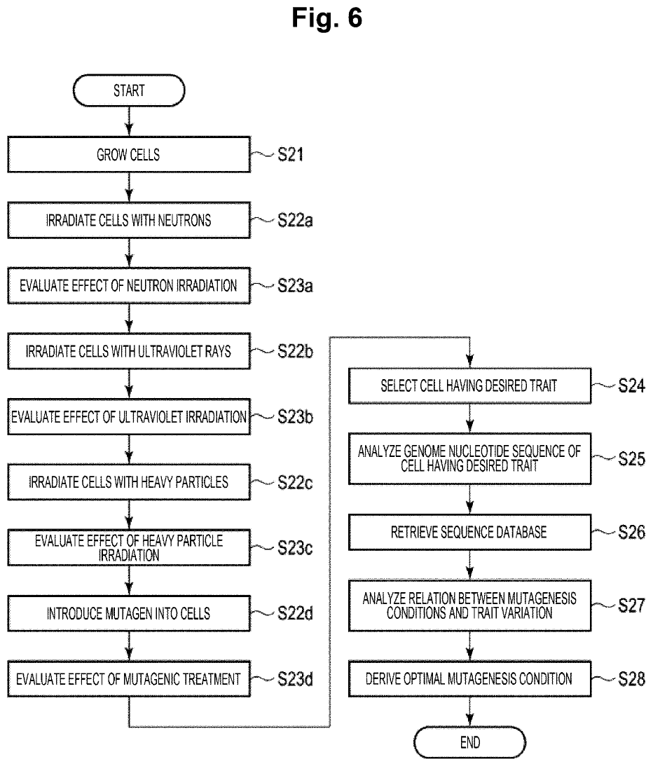

FIG. 6 is a flow chart illustrating procedures performed by the mutant creation system according to the second embodiment of the present invention.

First, in the second embodiment, in step S21, the cell growth device 401 is used to grow cells to be irradiated.

As illustrated in FIG. 5, in the second embodiment, the neutron irradiation effect evaluation device 403a, the ultraviolet irradiation effect evaluation device 403b, the heavy particle irradiation effect evaluation device 403c, and the mutagenic treatment effect evaluation device 403d which are devices for evaluating the effects of irradiation or treatment by growth rate analysis using the number of living cells or with an absorptiometer are set so that these devices are selected in parallel as mutation induction means. In other words, the various irradiation devices, the treatment device, and the various effect evaluation devices constitute a mutagenesis device group (symbol a in FIG. 5).

Using these devices, mutation induction in cells and the effect of irradiation or introduction are evaluated. That is, untreated cells are irradiated with neutrons by the neutron irradiation device 402a (step S22a), the effect of neutron irradiation on the cells is evaluated by the neutron irradiation effect evaluation device 403a (step S23a), untreated cells are irradiated with ultraviolet rays by the ultraviolet irradiation device 402b (step S22b), the effect of ultraviolet irradiation on the cells is evaluated by the ultraviolet irradiation effect evaluation device 403b (step S23b), untreated cells are irradiated with heavy particle beams by the heavy particle irradiation device 402c (step S22c), the effect of heavy particle beam irradiation on the cells is evaluated by the heavy particle irradiation effect evaluation device 403c (step S23c), a mutagen is introduced into untreated cells by the mutagenic treatment device 402d (step S22d), and the effect of mutagen introduction into the cells is evaluated by the mutagenic treatment effect evaluation device 403d (step S23d).

After these evaluations, in step S24, the cell selection device 404 is used to select a cell having a desired trait based on results of evaluations by the various effect evaluation devices.

The order of steps S22a and S23a related to the neutron irradiation, steps S22b and S23b related to the ultraviolet irradiation, steps S22c and S23c related to the heavy particle beam irradiation, and steps S22d and S23d related to the mutagenic treatment are not particularly limited, and these steps may be executed in any order. In other words, as long as the effect of irradiating neutrons, ultraviolet rays, or heavy particle beams is evaluated after, but not limited to immediately after, the irradiation and as long as the effect of introducing a mutagen is evaluated after, but not limited to immediately after, the introduction, the order of these steps may be changed.

In step S24, for example, the cell selection device 404 compares an evaluation of the effect of neutron irradiation on the cells grown by the cell growth device 401 and before treated by the corresponding subsequent device of the cell growth device 401, an evaluation of the effect of ultraviolet irradiation on the cells grown by the cell growth device 401 and before treated by the corresponding subsequent device, an evaluation of the effect of heavy particle beam irradiation on the cells grown by the cell growth device 401 and before treated by the corresponding subsequent device, and an evaluation of the effect of mutagen introduction into cells grown by the cell growth device 401 and before treated by the corresponding subsequent device, thereby using results of the comparison to select a cell having a desired trait.

Next, in step S25, the nucleotide sequence analyzer 405 is used to analyze a genome nucleotide sequence of the selected cell, that is, the cell having the desired trait.

The nucleotide sequence analyzer 405 supplies the analyzed genome nucleotide sequence to the sequence database retrieval device 406, and in step S26, the sequence database retrieval device 406 compares the analyzed genome nucleotide sequence and a nucleotide sequence of the cell before mutation which is stored in the sequence database. Accordingly, the mutated sequence in the genome nucleotide sequence caused by the mutation induction means is identified, which leads to identification of a mutation pattern, that is, a genome nucleotide sequence that has caused a trait variation in the cell.

Next, in step S27, the relation analysis device 407 is used to analyze the relation among the information on the identified mutated sequence, the mutagenesis conditions for the selected cell, and the genome nucleotide sequence that has caused the trait variation in the selected cell.

In step S28, based on a result of analysis by the relation analysis device 407, the mutagenesis condition derivation device 408 derives a mutagenesis condition by an optimal mutagenesis method that causes a desired trait variation and reflects the condition in the treatments by the mutagenesis device group, thereby creating a desired mutant.

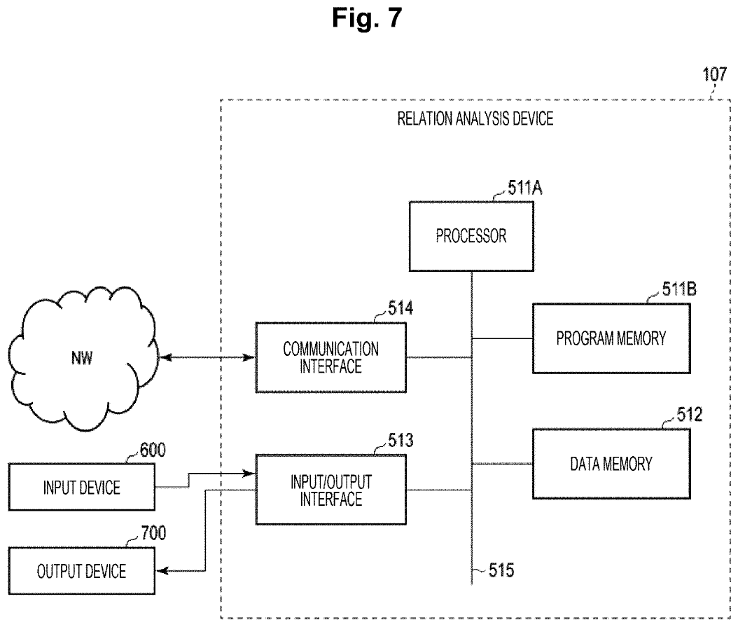

FIG. 7 is a block diagram illustrating a hardware configuration of a relation analysis device according to an embodiment of the present invention.

In the example illustrated in FIG. 7, the relation analysis device 107 according to the aforementioned embodiment is, for example, a server computer or a personal computer and includes a hardware processor 511A such as a CPU. To this hardware processor 511A, a program memory 511B, a data memory 512, an input/output interface 513, and a communication interface 514 are connected via a bus 515. Hereinafter, the relation analysis device 107 will be described as an example, but the other devices included in the mutant creation system such as the various irradiation devices, the treatment device, the various evaluation devices, the cell selection devices 104 and 404, the nucleotide sequence analyzers 105 and 405, the sequence database retrieval devices 106 and 406, and the relation analysis device 407 are configured in a similar manner.

The communication interface 514 includes, for example, one or more wireless communication interface units and transmits and receives information to and from a communication network NW. An example of a wireless interface used herein includes an interface that employs a low-power wireless data communication standard such as a wireless Local Area Network (LAN).

To the input/output interface 513, an input device 600 and an output device 700 are connected. The input device 600 and the output device 700 are attached to the relation analysis device 107 and operated by a user or the like.

The input/output interface 513 captures operation data inputted by a user or the like through the input device 600 such as a keyboard, a touch panel, a touchpad, and a mouse, outputs the data to the output device 700 including a display device using liquid crystals or organic Electro Luminescence (EL), and allows the output device 700 to display the output data. The input device 600 and the output device 700 may be embedded in the relation analysis device 107 or may be an input device and an output device of another information terminal that enables communications with the relation analysis device 107 over a network NW.

The program memory 511B is a non-transitory tangible storage medium in which, for example, a non-volatile memory enabling writing and reading at any time such as a Hard Disk Drive (HDD) and a Solid State Drive (SSD) and a non-volatile memory such as a Read Only Memory (ROM) are combined. The program memory 511B stores programs required for executing various types of control processing according to an embodiment.

The data memory 512 is a tangible storage medium in which, for example, the aforementioned non-volatile memory and a volatile memory such as a Random Access Memory (RAM) are combined and is used for storing various types of data acquired and generated during the course of various types of processing.

The relation analysis device 107 according to an embodiment of the present invention may be a data processing device that includes a processing functional unit using software.

An information storage unit used as a working memory or the like of the relation analysis device 107 may employ the data memory 512 illustrated in FIG. 7. However, these storage areas are not necessarily included in the relation analysis device 107 and may be areas in, for example, an external storage medium such as a Universal Serial Bus (USB) memory or a storage device such as a database server in the cloud.

The aforementioned processing functional unit is achieved by causing the hardware processor 511A to read and execute the programs stored in the program memory 511B. Note that the processing functional unit may be achieved in other various forms including an integrated circuit such as an Application Specific Integrated Circuit (ASIC) and a Field-Programmable Gate Array (FPGA).

In addition, the methods disclosed in the embodiments may be stored, as programs (software tool) to be executed by a computing machine (computer), in a recording medium such as a magnetic disk (Floppy (registered trademark) disk or hard disk), an optical disc (CD-ROM, DVD, or MO), and a semiconductor memory (ROM, RAM, or Flash memory) or may be distributed by being transmitted through a communication medium. Note that the programs stored in a medium also include a setting program for allowing the computing machine to implement a software tool (including not only an execution program but also a table and a data structure) to be executed by the computing machine. The computing machine that achieves the devices according to the embodiments reads the programs recorded in the recording medium, constructs a software tool by the setting program as needed, and controls operation by the software tool, thereby executing the aforementioned processing. Note that the recording medium herein is not limited to one used for distribution, and examples of the recording medium include a storage medium such as a magnetic disk and a semiconductor memory inside a computing machine or those disposed in a device connected to the computing machine over a network.

Note that the present invention is not limited to the embodiments, and various modifications can be made in the implementation phase without departing from the gist of the invention. Alternatively, the embodiments may be combined appropriately, and in that case, combined effects can be obtained. Furthermore, the embodiments include various inventions, and the various inventions may be extracted by a combination selected from a plurality of disclosed constituents. For example, as long as the problems are solved and the effects are obtained even though some constituents are deleted from all the constituents disclosed in the embodiments, a configuration from which the constituents are deleted may be extracted as an invention.

### REFERENCE SIGNS LIST

101 ALGAE CULTURE DEVICE
102 CENTRIFUGE TUBE
103, 402a NEUTRON IRRADIATION DEVICE
104, 404 CELL SELECTION DEVICE
105, 405 NUCLEOTIDE SEQUENCE ANALYZER
106, 406 SEQUENCE DATABASE RETRIEVAL DEVICE
107, 407 RELATION ANALYSIS DEVICE
401 CELL GROWTH DEVICE
402b ULTRAVIOLET IRRADIATION DEVICE
402c HEAVY PARTICLE IRRADIATION DEVICE
402d MUTAGENIC TREATMENT DEVICE
403a NEUTRON IRRADIATION EFFECT EVALUATION DEVICE
403b ULTRAVIOLET IRRADIATION EFFECT EVALUATION DEVICE
403C HEAVY PARTICLE IRRADIATION EFFECT EVALUATION DEVICE
403d MUTAGENIC TREATMENT EFFECT EVALUATION DEVICE
408 MUTAGENESIS CONDITION DERIVATION DEVICE

1. A mutant creation apparatus, the apparatus comprising:
a circuitry configured to:
obtain mutants of an organism by irradiating the organism, or a target for creating a mutant, with a radiation or electromagnetic energy for inducing a mutation in a nucleotide sequence that causes a trait variation in the organism or introducing a chemical substance into the organism for inducing the mutation;
select a mutant having a desired trait from mutants of the organism;
analyze a nucleotide sequence of the selected mutant;
identify a nucleotide sequence that provides the desired trait of the mutant in the analyzed nucleotide sequence; and
analyze a condition under which a desired mutant of the organism is created based on a result of identification.

2. The mutant creation apparatus according to claim 1, the apparatus further comprising a storage device that stores a nucleotide sequence of the organism before obtaining mutants of the organism;
wherein the circuitry is configured to:
compare the analyzed nucleotide sequence and the nucleotide sequence stored in the storage device to identify a nucleotide sequence that provides the desired trait of the mutant.

3. The mutant creation apparatus according to claim 1,
wherein circuitry is configured to:
identify a mutated sequence of mutants of the organism in the analyzed nucleotide sequence to identify a nucleotide sequence that causes a change for the desired trait of the mutant.

4. The mutant creation apparatus according to claim 1,
wherein circuitry is configured to:
treat the organism using any of a plurality of methods for inducing a mutation in a nucleotide sequence that causes a trait variation in the organism,
evaluate a result of treatment to obtain mutants of the organism, and
select a mutant having a desired trait from mutants of the organism using a result of evaluation.

5. A mutant creation method performed by a mutant creation apparatus, the method comprising:
obtaining mutants of an organism by irradiating the organism, or a target for creating a mutant, with a radiation or electromagnetic energy for inducing a mutation in a nucleotide sequence that causes a trait variation in the organism or introducing a chemical substance into the organism for inducing the mutation;
selecting a mutant having a desired trait from mutants of the organism;
analyzing a nucleotide sequence of the selected mutant;
identifying a nucleotide sequence that provides the desired trait of the mutant in the analyzed nucleotide sequence; and
analyzing a condition under which a desired mutant of the organism is created based on a result of identification.

6. The mutant creation method according to claim 5,
wherein the mutant creation apparatus further comprises a storage device that stores a nucleotide sequence of the organism before obtaining mutants of the organism, and
compare the analyzed nucleotide sequence and the nucleotide sequence stored in the storage device to identify a nucleotide sequence that provides the desired trait of the mutant.

7. The mutant creation method according to claim 5,
wherein identify a mutated sequence in the analyzed nucleotide sequence to identify a nucleotide sequence that causes a change for the desired trait of the mutant.

8. A non-transitory computer readable storage medium storing a program, when executed by a mutant creation apparatus to provide the steps of:
obtaining mutants of an organism by irradiating the organism, or a target for creating a mutant, with a radiation or electromagnetic energy for inducing a mutation in a nucleotide sequence that causes a trait variation in the organism or introducing a chemical substance into the organism for inducing the mutation;
selecting a mutant having a desired trait from mutants of the organism;
analyzing a nucleotide sequence of the selected mutant;
identifying a nucleotide sequence that provides the desired trait of the mutant in the analyzed nucleotide; and
analyzing a condition under which a desired mutant of the organism is created based on a result of identification.
